(19) United States
(12) Patent Application Publication
(10) Pub. No.: US 2019/0015146 A1
(43) Pub. Date: Jan. 17, 2019

(54) Devices and Methods for Endoscopic Cryogenic Surgery

(71) Applicant: Brian R. DuBois, Redwood City, CA (US)

(72) Inventor: Brian R. DuBois, Redwood City, CA (US)

(21) Appl. No.: 16/034,827

(22) Filed: Jul. 13, 2018

Related U.S. Application Data
(60) Provisional application No. 62/532,259, filed on Jul. 13, 2017.

(51) Int. Cl. A61B 18/02

(52) U.S. Cl. CPC A61B 18/0218; A61B 2017/00269

(57) ABSTRACT

An exemplary medical apparatus includes a handle including a proximal knob at its distal end, a reservoir of cryogenic fluid in the handle, a shaft extending distally from the handle, the shaft configured to convey cryogenic fluid from the reservoir to a distal end of the shaft; where the shaft comprises a malleable material, and a rotating tip at a distal end of the shaft, where rotation of the proximal knob causes rotation of the rotating tip about an axis of the shaft.

This application claims priority to U.S. Provisional Patent Application Ser. No. 62/532,259, filed on Jul. 13, 2017, which is herein incorporated by reference in its entirety.

### FIELD OF THE INVENTION

The invention generally relates to devices and methods for cryogenic surgery.

### BACKGROUND

Endoscopic Sinus Surgery

Endoscopic sinus surgery (ESS) is the time-honored technique for the management of chronic rhinosinusitis (CRS). Nevertheless, between 8% and 38% of patients treated with ESS develop recurrent symptoms that require revision surgery. Synechia development occurs as a result of the juxtaposition of two injured mucosal surfaces. Following ESS, remaining synechia between the middle turbinate and the lateral nasal wall might cause narrowing of the sinus outflow tracts. Common findings first described by Ramadan et al. during revision ESS included synechia in 56% of patients and stenosis of either the maxillary (27% of cases) or frontal ostium (25% of patients). However, the most recent multi-institutional study by Henriquez et al. demonstrated that about 20% of patients had evidence of synechia following ESS.

Proper healing that results in regenerated mucosa rests on optimal wound healing. Disruption of normal healing may result in granulation, adhesion, crust formation, and infection. Following surgery, fibrous tissue may overgrow on opposed damaged mucosal surfaces producing synechia. An extensive adhesion positioned near sinus ostium hampers drainage and ventilation with subsequent recurrent infection. Some previous studies attempted to prevent synechia formation after ESS using topical mitomycin C. Although efficient in experimental settings, clinical studies reported limited success rates.

Endoscopic spray cryotherapy using low-pressure liquid nitrogen is a more recent technique used in the management of premalignant (Barrett's esophagus) and malignant diseases of the esophagus; its utility in sinus surgery was demonstrated by Albu et. al. as described below. Spray cryotherapy represents an alternative method in improving wound healing and diminishing adhesion formation. During the initial phase, cryotherapy induces disruption of cell membranes, vasoconstriction, endothelial damage, thrombosis, and ischemia. Nonetheless, cryotherapy induces vascular proliferation and, paradoxically, slows the healing processes when compared to healing after mechanical injuries. Spray cryotherapy snap-freezes the tissue through contact with droplets of liquid nitrogen, and induces immediate cell death. Even though the grade of cell loss is comparable to either radiofrequency or argon plasma ablation which apply energy to the tissue, spray cryotherapy conversely extracts heat energy from the target tissue. This cooling effect protects the tissue architecture and extracellular matrix, generating a favorable wound response and decreased scarring. A recent multicenter, retrospective cohort study demonstrated that endoscopic spray cryotherapy is a safe and well-tolerated therapy for esophageal cancer staged T1 to T4. Complete treatment of intraluminal disease was noted in 61% overall and 75% of mucosal cancers. No serious adverse effects and reduced complication rate were reported. Taking into account the promising results previously described, spray cryotherapy has been successfully employed in various diseases such as glottic and subglottic stenosis, carcinoma of the pleura, and radiation proctitis. Recently, spray cryotherapy was also employed in the course of bronchoscopy in expectation of lung resection. Histologic examination of the resected specimens described cellular necrosis limited to the mucosa and submucosa without any damage to the underlying connective tissue.

Because the development of synechia and granulations is noticeable commonly during the fifth to seventh postoperative day, the action of an antisynechia agent should be most pronounced over this period. In an experimental model of CRS in the rabbit, Gocea et al. demonstrated that in the local inflammatory background, cryotherapy prompts local necrosis during the first week, and wound healing is characterized by improved organization of collagen fibers. Moreover, more areas with normally ciliated epithelia were found in the cryotherapy-treated group. It is well-known that improved wound healing and re-epithelialization minimizes the risk of adhesion and infection.

Counting on the safety and feasibility demonstrated in these experiments, the antiadhesive properties of cryotherapy following ESS were tested by Albu et. al. in a clinical trial. Albu et. al. sought to assess the influence of spray cryotherapy on wound healing following ESS. The therapy included four cycles of 5-second spray cryotherapy with a complete thaw of the treated area between each application. Spray cryotherapy was performed with the Brymill (Ellington, Conn.) CRY-AC-3 Cryogenic System, a device commonly used in cryosurgery for common skin conditions. This device dispenses liquid nitrogen through a straight, rigid shaft, and the spray is directed in line with the axis of the shaft. The Albu et. al. trial demonstrated that intraoperative cryotherapy accompanied by nasal irrigation and topical corticosteroids is associated with statistically significant improved objective postoperative middle meatus appearance. The objective outcome measures were represented by the Lund-Kennedy and POSE scores. The use of the POSE scoring system allows the gathering of a larger amount of data for healing assessment, including crusting, mucosal edema, discharge, synechia, and granulation formation. Cryotherapy is associated with a significant reduced rate of adhesions of the middle turbinate, edema, scarring, and stenosis of ostium during the follow-up period. There were no subjective changes in olfactory function reported. The results of the Albu et. al. clinical study reveal a significant improvement in postoperative objective scores demonstrating significant enhanced healing following spray cryotherapy following ESS.

The use of liquid nitrogen in surgery requires special safety and handling considerations which limit its use. It is extremely cold (−196° C.), which means that even very small amounts will cool any surface that it contacts. Additionally, liquid nitrogen must be allowed to vent as it evaporates to prevent the buildup of pressure. As a result, the reservoir of liquid nitrogen must be periodically replenished. Finally, although nitrogen is the most abundant gas in the atmosphere, excessive amounts of nitrogen displaces oxygen and can cause asphyxiation when a room containing evaporating nitrogen is not properly ventilated.

As stated above, the Albu study was conducted using the Brymill (Ellington, Conn.) CRY-AC-3 Cryogenic System, a device commonly used in cryosurgery for common skin conditions (FIG. 1). This device dispenses liquid nitrogen through a straight, rigid shaft and the spray is directed in line with the axis of the shaft. However, this configuration limits the areas within the nasal passageway and sinuses where the cryogenic fluid can be administered. The straight, rigid shaft enables the operator to spray only the areas of the nasal passageways that are in the “line of sight” through the nares. The operator is unable to access areas that require a curved shaft such as the maxillary sinuses, frontal recess, ethmoid sinuses, or other areas of the nasal cavity. Furthermore, if the cryogenic spray is directed distally in the direction along the axis of the shaft, mucosa that is at an angle to the shaft is difficult to spray, i.e., the operator cannot spray the cryogenic fluid at an angle to the centerline of the shaft to spray behind structures or normal to the walls of the nasal passageway.

It is for these reasons that adoption of spray cryotherapy has been limited and an opportunity exists to improve utility, safety, and convenience.

Tonsil Stones

Tonsil stones (tonsilloliths) are soft aggregates of bacterial and cellular debris that form in the tonsillar crypts, the crevices of the tonsils. While they occur most commonly in the palatine tonsils, they may also occur in the lingual tonsils. Tonsil stones are common and occur in approximately 10% of the population. They may be a nuisance and difficult to remove, but are usually not harmful, although they are one of the causes of bad breath and always give off a putrid smell. Recently, an association between biofilms and tonsilloliths was shown. Central to the biofilm concept is the assumption that bacteria form a three dimensional structure, dormant bacteria being in the center to serve as a constant nidus of infection. This impermeable structure renders the biofilm immune to antibiotic treatment.

Decreasing the surface area of the crypts, crevices, etc. of the tonsils has been performed via laser resurfacing, a procedure called laser cryptolysis using a local anesthetic. A scanned carbon dioxide laser selectively vaporizes and smoothes the surface of the tonsils. This technique flattens the edges of the crypts and crevices that collect the debris, preventing trapped material from forming stones. However, the high cost of the laser and pain subsequent to the procedure are factors that affect the practicality of the procedure, and limit usage of the procedure to the most extreme cases. Spray cryotherapy is an effective method of destroying biofilms and resurfacing the tonsils to reduce the propensity of the crypts and crevices to collect debris and forming a nidus for tonsil stones. However, the same drawbacks of the use of liquid nitrogen for spray cryotherapy for endoscopic sinus surgery are present when liquid nitrogen is used for spray cryotherapy for tonsil resurfacing.

The same healing principles apply to other areas of surgery where it is desirable to reduce scarring and adhesions. Cardiac bypass surgery causes significant scarring and adhesions because of the difficulty in closing the pericardium after the bypass has been completed. Orthopedic surgery involving knee joint replacements results in scarring that affects patient mobility. Laparoscopic surgeries result in scarring and adhesions that cause discomfort and complications. Traumatic injuries result in scarring and adhesions that can affect patient comfort and function. Spray cryotherapy can be useful in these situations, but has the drawbacks described above.

Accordingly, there is need for improved methods and apparatus for use in surgical procedures where reduction of scarring and/or adhesions is desired.

### SUMMARY

One embodiment of a medical apparatus includes a handle, a reservoir of cryogenic fluid in the handle; and a shaft extending distally from the handle, the shaft configured to convey cryogenic fluid from the reservoir to a distal end of the shaft; wherein the shaft includes a malleable material.

One embodiment of a method for treating tissue of a patient includes possessing a device comprising a handle, a reservoir of cryogenic fluid in the handle, and a shaft extending distally from the handle, the shaft configured to convey cryogenic fluid from the reservoir to a distal end of the shaft, where the shaft includes a malleable material; apposing the distal end of the shaft to tissue of a patient; causing cryogenic fluid to travel from said handle to said distal end of said shaft; and allowing the cryogenic fluid to expand to a gas phase upon exiting the distal end of the shaft, where the gas phase cryogenic fluid is at a cryogenically therapeutic temperature.

The characteristics and utilities of the present invention described in this summary and the detailed description below are not all inclusive. Many additional features and advantages will be apparent to one of ordinary skill in the art given the following description. There has thus been outlined, rather broadly, the more important features of the invention in order that the detailed description thereof that follows may be better understood, and in order that the present contribution to the art may be better appreciated.

### BRIEF DESCRIPTION OF THE DRAWINGS

FIG. 1 is a perspective view of a cryogenic gas treatment tool.

FIG. 2 is a cross-section view of a handle of the cryogenic gas treatment tool of FIG. 1.

FIG. 3 is a cross-section view of the distal end of a shaft of the cryogenic gas treatment tool of FIG. 1.

FIG. 4 is a cross-section view of the distal end of the handle of FIG. 2 with a proximal end of an interchangeable shaft.

FIG. 5 is a cross-section view of a shaft of the cryogenic gas treatment tool of FIG. 1 with a first nozzle.

FIG. 6 is a cross-section view of a shaft of the cryogenic gas treatment tool of FIG. 1 with a second, right-angle nozzle.

FIG. 7 is a cross-section view of a shaft of the cryogenic gas treatment tool of FIG. 1 with a paddle at the distal end thereof.

FIG. 8 is a cross-section view of a shaft of the cryogenic gas treatment tool of FIG. 1 with a curved end effector at the distal end thereof.

FIG. 9 is a cross-section view of a shaft of the cryogenic gas treatment tool of FIG. 1 with a rotating end effector at the distal end thereof.

FIG. 10 is a cross-section view of a nasal passageway and frontal recess of a patient, showing a portion of the shaft of the cryogenic gas treatment tool of FIG. 1 that has been shaped to access the frontal recess, with the rotating end effector of FIG. 9 rotated to spray anteriorly.

FIG. 11 is a cross-section view of a nasal passageway and frontal recess of a patient, showing a portion of the shaft of the cryogenic gas treatment tool of FIG. 1 that has been shaped to access the frontal recess, with the rotating end effector of FIG. 9 rotated to spray posteriorly.

FIG. 12 is a cross-section view of a nasal passageway and frontal recess of a patient, showing a portion of the shaft of the cryogenic gas treatment tool of FIG. 1 that has been shaped to access the frontal recess, with the rotating end effector of FIG. 9 rotated to spray superiorly.

The use of the same reference symbols in different figures indicates similar or identical items.

### DETAILED DESCRIPTION

The present invention includes devices and methods that improve safety and convenience of cryotherapy in endoscopic or laparoscopic applications such as, but not limited to sinus surgery, turbinate reduction, posterior nasal nerve destruction, and any other surgical location where it is desirable to cause cell death within 3 mm of the tissue surface. The methods and devices herein may be useful in, for example, any procedure in which an internal cavity or orifice sustains tissue injury. The methods and devices herein may also be useful when freezing or cell death of the tissue is desired, although in the preferred embodiments permanent tissue damage is avoided.

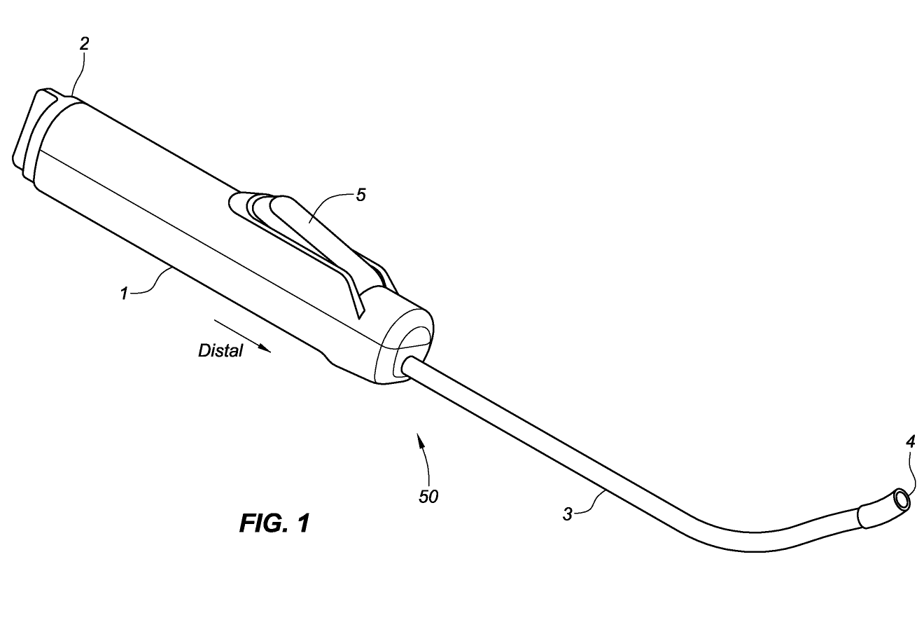

Referring to FIG. 1, a cryogenic gas treatment tool 50 includes a handle 1 and a shaft 3 extending from the distal end of the handle 1. Advantageously, the handle 1 is ergonomically designed to increase comfort of and control by the clinician. A nozzle 4 is located at the distal end of the shaft 3.

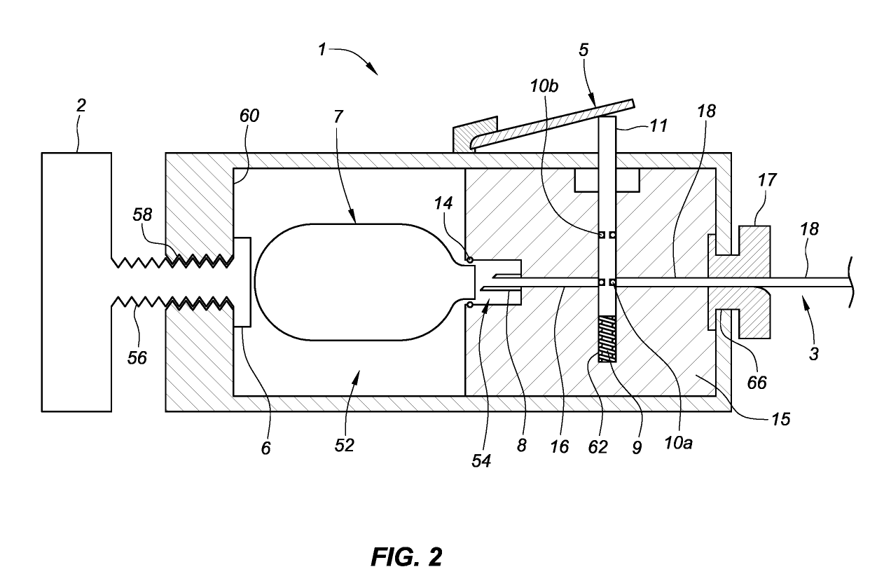

Referring also to FIG. 2, the handle 1 includes a cavity 52 defined therein in which a reservoir 7 is located. Initially, prior to actuation of the cryogenic gas treatment tool 50, the reservoir 7 advantageously is sealed. The reservoir 7 may be filled with biocompatible cryogenic fluid, such as but not limited to CO₂, nitrous oxide, nitrogen, or argon. A mixture of fluids may be used, rather than a single fluid. Initially, the fluid in the reservoir 7 may be compressed to the point at which it is liquid at room temperature. Alternately, the fluid in the reservoir 7 may be compressed gas that is not in a liquid state. In use, as fluid escapes the reservoir 7, the fluid in the reservoir 7 may change from a liquid to a gaseous state. The reservoir 7 includes a narrow end 7a that sits partially within a recess 54 in a valve body 15, with the narrow end 7a in proximity to or in contact with a pierce 8. The narrow end 7a of the reservoir 7 may be slightly smaller in diameter and/or cross-section than the diameter and/or cross-section of the recess 54, such that the narrow end 7a of the reservoir 7 is held securely within the recess 54. A cartridge seal 14 may be located in the recess 54, and may be an O-ring or similar seal that receives the narrow end 7a of the reservoir 7 and prevents or reduces gas leakage out of the recess 54 in use. Alternately, the reservoir 7 may have any other suitable shape that allows for fluid to be stored, and then to be released at a selected time. Proximal to the reservoir 7, a knob retention flange 6 is located in proximity to or in contact with the proximal end of the reservoir 7. The knob retention flange 6 may be connected to a threaded rod 56 that is held within and that is rotatable within a threaded receiver 58 that is defined in a proximal wall 60 of the handle 1. The knob retention flange 6 is wider than the threaded receiver 58, thereby preventing the threaded rod 56 against inadvertently being unscrewed from the handle 1; contact between the knob retention flange 6 and the portion of the proximal wall 60 surrounding the distal opening of the threaded receiver 58 stops further proximal motion of the threaded rod 56. A knob 2 may be connected to the proximal end of the threaded rod 56. As described in greater detail below, rotation of the knob 2 advances the threaded rod 56 and urges the reservoir 7 distally, thereby urging the narrow end 7a of the reservoir 7 into the pierce 8, which in turn punctures the narrow end 7a of the reservoir 7 and allows fluid outflow from the narrow end 7a of the reservoir 7.

The reservoir 7 may be fixed in position within the handle 1, such that the handle 1 (and thus the cryogenic gas treatment tool 50) is actuable for a single use with a single reservoir 7. Optionally, the handle 1 may be configured to allow replacement of the reservoir 7. Such replacement may be useful where treatment of a single patient requires more fluid than can be held in a single reservoir 7. Further, the handle 1 may be configured to be reusable, such that it is sterilizable and/or cleanable between uses, and the reservoir 7 is thus exchanged between uses. If so, the knob 2 and the proximal wall 60 of the handle 1 may be modified to allow the spent reservoir 7 to be removed proximally from the handle 1, and to allow a fresh reservoir 7 to be added into the cavity 52. As another embodiment, the handle 1 may include a door (not shown) in its side that can be opened to the cavity 52 and allow the spent reservoir 7 to be removed laterally from the handle 1, and to allow a fresh reservoir 7 to be added into the cavity 52.

Distal to the pierce 8 is a valve body 15. A proximal cryogenic fluid path 16 extends from the pierce 8 to a valve seat 62. Within the valve seat 62 sits a valve 11. The valve 11 may be generally cylindrical, but may have any other suitable shape or cross-section. Similarly, the valve seat 62 has a corresponding shape that may be generally cylindrical, but may have any other suitable shape or cross-section. One end of the valve 11 extends outward from the handle 1 and engages a trigger 5. The other end of the valve 11 engages a spring 9 that sits at the bottom of the valve seat 62. The spring 9 may be fixed to the valve seat 62, or may be floating free between the bottom of the valve seat 62 and the valve 11, trapped in place therebetween. Two O-rings 10 are positioned on the valve 11. Advantageously, both O-rings 10 have a square cross-section. Alternately, at least one O-ring 10 may have a different cross-section. The lower O-ring 10a, closer to the spring 9, is positioned to block outflow from the proximal cryogenic fluid path 16 when the trigger 5 is in a neutral position. The trigger 5 may be biased to the neutral position, such as by a spring (not shown) that biased the trigger 5 away from the valve 11. The upper O-ring 10b is spaced apart from the lower O-ring 10a a distance as least as far as the height of the proximal cryogenic fluid path 16. Alternately, the O-rings 10 may be spaced apart a different distance. A conduit 18 extends distally from the valve seat 62, and advantageously is coaxial with the proximal cryogenic fluid path 16. In some embodiments, the conduit 18 extends from the valve seat 62 to the distal end of the shaft 3, and is a flexible tube with an inner diameter of substantially 0.010 inches. According to other embodiments, the conduit 18 has a larger or smaller inner diameter. The conduit 18 may be fabricated from any suitable material that is configured to withstand exposure to cryogenic fluid; such materials appropriate in a medical setting will be recognized by those skilled in the art. According to other embodiments, the conduit 18 does not extend all the way to the valve seat 62, and instead a path is defined through the valve body 15 to a point at which the conduit 18 is affixed to that valve body 15. As described in greater detail below, when the trigger 5 is depressed or otherwise actuated, the valve 11 moves against the bias of the spring 9, moving the lower O-ring 10a out of its initial position in which it blocked outflow from the proximal cryogenic fluid path 16 and inflow into the conduit 18. Fluid is then free to flow out of the proximal cryogenic fluid path 16, into the valve seat 62 between the O-rings 10, and then out of the valve seat 62 into the conduit 18.

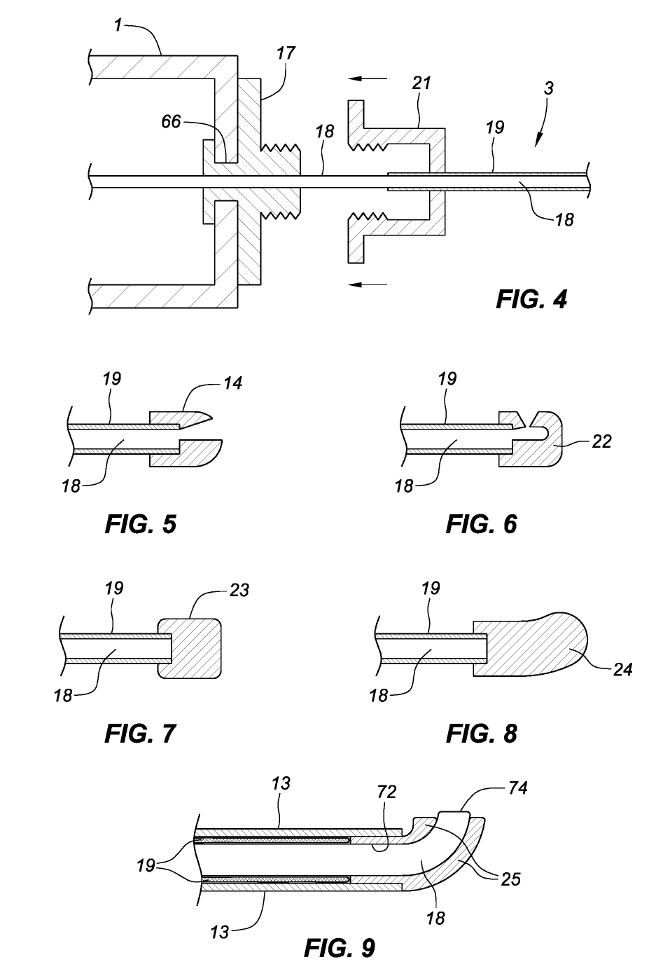

Referring also to FIG. 4, a proximal knob 17 is located at the distal end of the handle 1. The proximal knob 17 extends through an aperture 66 at the distal end of the handle 1, and is rotatable freely within that aperture 66. The proximal and distal ends of the proximal knob 17 are wider than the aperture 66, in order to hold a portion of the proximal knob 17 within the aperture 66. The conduit 18 extends through the proximal knob 17. The conduit 18 may be a tube that is affixed to the valve body 15 and that passes through the proximal knob 17, such that the proximal knob 17 is configured to rotate about that tube that is the conduit 18. Alternately, the proximal end of the proximal knob 17 is sealed relative to the valve body 15 sufficiently such that the conduit 18 is a passage through the valve body 15 and the proximal knob 17, and a tube extends distally from the proximal knob 17. A distal knob 21 is configured to mate with the proximal knob 17, such as through a threaded rotational connection, a quarter turn connection, a bayonet connection, or any other suitable connection. The distal knob 21 is connected to a malleable component 19; for example, the proximal end of the malleable component 19 is affixed to the distal knob 21. The nozzle 4, rotating tip 25, or other end effector is fixed to the opposite end of the malleable component. The malleable component 19 has a lumen defined therethrough, where the inner diameter of that lumen is sized to receive the outer diameter of the conduit 18, as described in greater detail below. The malleable component 19 is fabricated from malleable material such as, but not limited to, annealed steel wire or tubing. The types of malleable material appropriate in a medical setting will be recognized by those skilled in the art.

The shaft 3 is formed by the combination of the conduit 18 and the malleable component 19 substantially coaxial with the conduit. The conduit 18 is a tube through which cryogenic fluid can flow. The malleable component 19 extends substantially along the length of the conduit 18 and substantially surrounds the conduit 18 generally along the length of the conduit 18. When the distal knob 21 is first placed onto the conduit 18, the proximal end of malleable cover 19 is placed against the distal end of the conduit 18, and the distal knob 21 is then moved proximally to pull the malleable cover 19 over and along the conduit 18. The distal knob 21 is then connected to the proximal knob 17. In this way, the proximal end of the malleable component 19 slides over the conduit 18 and the shaft 3 is ready for use. The fit between the malleable component 19 and the conduit 18 is loose enough to allow the conduit 18 to slide into the proximal end of the malleable component 19. The malleable component 19 is bent outside the body to conform to the anatomy to be treated, as described in greater detail below. Alternately, the malleable component 19 may be replaced in whole or in part by a rigid cover.

The connection between the distal knob 21 and proximal knob 17 allows for a plurality of different shafts 3, and therefore nozzles 4, to be selected and used by a clinician, depending on the individual clinical needs of a patient. Advantageously, the distal knob 21 is detachable from the proximal knob 17 after connection, in order to allow the clinician to change the shaft 3 (and therefore nozzle) being used during a procedure, or between procedures where either the entire shaft 3 and distal knob 21, or a portion of the shaft 3, is disposable and at least a portion of the handle 1 is reusable. However, the connection between the distal knob 21 and the proximal knob 17 may be permanent, such that the distal knob 21 and the proximal knob 17 cannot be disengaged by the clinician after their connection.

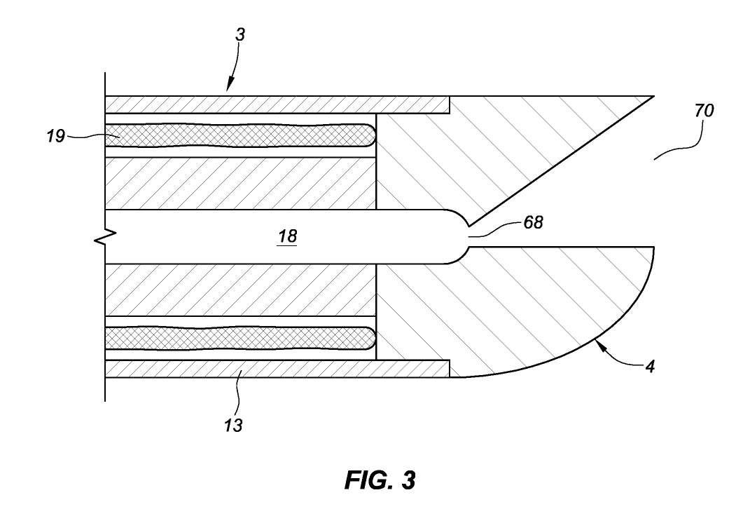

As described above, a plurality of shafts 3 may be available to the clinician, each associated with a different nozzle 4. Each nozzle 4 may be configured for a different spray pattern for cryogenic fluid. Each nozzle 4 begins with a small aperture 68 at the distal end of the conduit 18 Referring to FIG. 3, the nozzle 4 is configured to diffuse the cryogenic fluid in a cone-shaped pattern. The nozzle 4 of FIG. 3 has a cone-shaped pattern that is at an angle to the shaft 3, such that the nozzle 4 has a spray pattern that is off-axis relative to the shaft 3. The off-axis spray pattern may allow the clinician to better visualize the spray pattern, and to spray at an angle to the axis of the shaft 3 to more conveniently spray the treatment site. Alternately, the nozzle 4 may be configured to have a cone-shaped spray pattern that is substantially aligned with the axis of the shaft 3. Alternately, the nozzle 4 may be configured to generate a spray pattern having a shape different from a cone. Alternately, the nozzle 4 may be configured to generate a non-uniform flow and/or shape of cryogenic fluid. Alternately, as described with regard to embodiments below, the nozzle 4 may be omitted, and the cryogenic fluid may flow directly out of the distal end of the conduit 18 such as shown in FIG. 9. As seen in FIG. 3, the malleable component 19 may be covered by a sheath 13, which is fabricated from flexible material that generally conforms to the outer surface of the malleable component 19. Alternately, the malleable component 19 may be replaced by the sheath 13. Referring to FIG. 5, the nozzle 4 is similar to that of FIG. 3, and the sheath 13 present in FIG. 3 is omitted. Referring to FIG. 6, a right angle nozzle 22 is shown. The right angle nozzle 22 opens at an angle generally perpendicular to the shaft 3, and may disperse cryogenic fluid in a cone-shaped pattern or other pattern. The sheath 13 is omitted from this configuration of nozzle 4, but may be included if desired.

Referring to FIG. 7, an end effector configured as a paddle 23 may be located at the distal end of the shaft 3. The paddle 23 may be a generally-closed chamber that allows for expansion of cryogenic fluid therein, thereby cooling the paddle 23 via the Joules-Thompson effect. The paddle 23 may be generally rectangular in shape, or may have a different shape. The paddle 23 may be fixedly attached to the sheath 13, thereby enabling it to be rotated relative to the conduit 18 and/or malleable component 19, or it may be fixedly attached directly to the malleable component 19 or to the conduit 18. The evaporated cryogenic gas may escape from the paddle 23 through a space in the shaft 3, such as the space between the malleable component 19 and to the conduit 18 and into the handle 1 of the device. Alternately, the paddle 23 may be configured to allow the evaporated cryogenic gas to escape from the paddle 23 through one or more openings on sides or edges of the paddle 23. Alternately, the paddle 23 may be configured to allow the cryogenic fluid to escape from the paddle 23 through one or more porous surfaces on sides or edges of the paddle 23 and evaporate as it escapes from the porous surfaces on the paddle 23.

The paddle 23 may be configured to spray the cryogenic fluid from one or more apertures in one or more surfaces such as the edges or sides of the paddle 23. The paddle 23 may be configured to minimize the cooling effect on certain surfaces to prevent inadvertent cooling of tissue they may contact, such as by insulating a part of the paddle 23 or preventing flow of cryogenic fluid into part of the paddle 23. For example, when freezing the mucosa in the proximity of the posterior nasal nerves, it may be necessary to place one surface of the paddle 23 against the mucosa in the proximity of the posterior nasal nerve, while another surface of the paddle 23 may be in contact with the lateral surface of the middle turbinate. While it is desired to cool the mucosa in the proximity of the posterior nasal nerve, it is not necessary to cool tissue on the middle turbinate and thermal insulation on the surface of the paddle 23 in contact with the middle turbinate reduces the likelihood of inadvertent cooling of that tissue.

Referring to FIG. 8, a curved end effector 24 may be located at the distal end of the shaft 3. The curved end effector 24 may be a generally-closed chamber that allows for expansion of cryogenic fluid therein, and may be similar to the paddle 23 described above, with a different shape. The curved end effector 24 may be curved in any manner, and have any suitable perimeter; the specific curvature advantageously relates to the specific clinical therapy to be performed by the curved end effector 24.

Referring to FIG. 9, a rotating tip 25 may be provided at the distal end of the shaft 3. The rotating tip 25 may be fixedly attached to and held in position by the sheath 13. The proximal end of the rotating tip 25 may be located on or near the centerline of the shaft 3, such that the proximal end of the rotating tip 25 is fixed to the sheath 13 and/or malleable component 19. Referring to FIG. 4, the sheath 13 and/or malleable component 19 are fixed to the distal knob 21 at or near their proximal end. Thus, rotation of the distal knob 21 causes rotation of the sheath 13 and/or malleable component 19 about their longitudinal axes. The distal end of the rotating tip 25 may be located away from the centerline of the shaft 3, and may be oriented away from the centerline of the shaft 3. The rotating tip 25 includes a channel 72 defined therein. The rotating tip 25 may receive the conduit 18 within the channel 72, such that the distal end 74 of the conduit 18 is substantially coterminous with the end of the channel 72. The conduit 18 may be fixed relative to the channel 72. Therefore, the distal end 74 of the conduit 18 rotates about the axis of the shaft 3 as the rotating tip 25 is rotated. The distal end 74 of the conduit 18 points radially away from the centerline of the shaft 3, allowing cryogenic fluid to exit the conduit 18 at an angle to the centerline of the shaft 3. Where the rotating tip 25 is used, cryogenic fluid expands as it escapes the distal end of the conduit 18, without being shaped by a nozzle 4. Alternately, a selected nozzle 4 as described above may be connected to or otherwise provided on the rotating tip 25. Optionally, the distal end 74 of the conduit 18 is located proximal to the end of the channel 72, such as at the proximal end of the rotating tip 25, such that gas passes out of the conduit 18 and through the channel 72 of rotating tip 25.

Operation

The operation of the cryogenic gas treatment tool 50 now will be described. Referring to FIG. 1, a user selects one of the shafts 3 that is suitable for the procedure to be performed. For this example, referring to FIG. 9, the user selects the shaft 3 with the rotating tip 25. Referring also to FIGS. 3-4, when the user first places the distal knob 21 onto the conduit 18, the proximal end of malleable cover 19 is placed against the distal end of the conduit 18, and the distal knob 21 is then moved proximally to pull the malleable cover 19 over and along the conduit 18. The user then couples the distal knob 21 to the proximal knob 17, such as by screwing them together, resulting in a complete shaft 3. Alternately, the user takes in hand a cryogenic gas treatment tool 50 in which a shaft 3 already has been coupled to the handle 1.

The user then bends the shaft 3 in a manner that facilitates advancement of the distal end of the shaft 3 to the particular site to be treated. The malleable component 19 is malleable, meaning that the shaft 3 holds a first shape prior to being bent by hand, is bendable to a second shape by hand, and then holds the second shape after the user has bent it. Where the shaft does not include a malleable component 19, the shaft 3 is not bent by the user, or may be bent in a different manner.

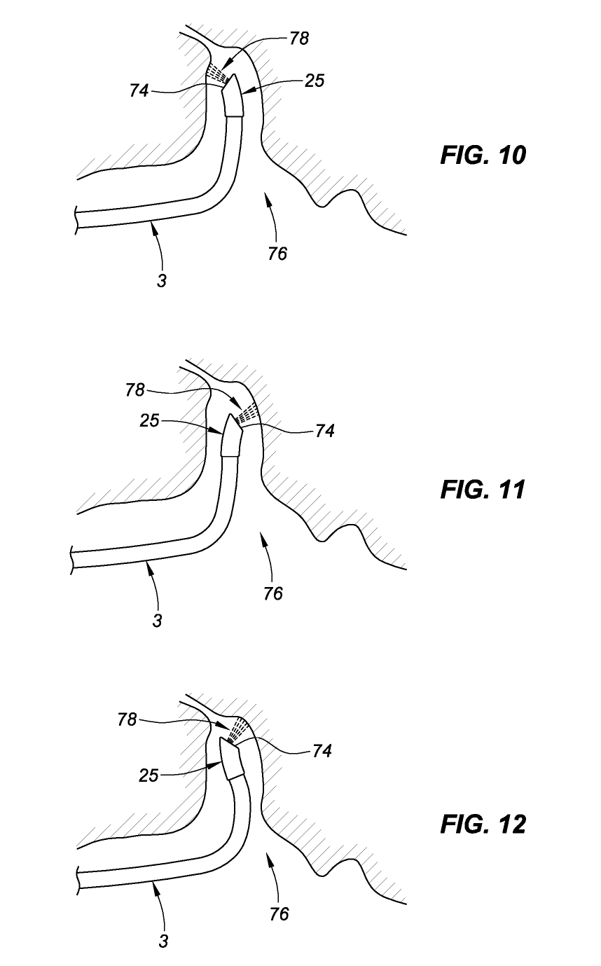

The user then actuates the knob 2 to urge the reservoir 7 toward the pierce 8. In one embodiment, rotation of the knob 2 advances the threaded rod 56 and urges the reservoir 7 distally. Continued advancement of the reservoir 7 causes the pierce 8 to puncture the distal end of the reservoir 7, allowing fluid to flow outward from the reservoir 7 into the proximal cryogenic fluid path 16. The distal end of the proximal cryogenic fluid path 16 is blocked by the lower O-ring 10a. Referring also to FIG. 10, the user inserts the shaft 3 into the patient's nasal cavity 76 through a nostril and advances the distal end of the shaft 3 to the treatment site. The user may directly visualize the treatment site, or may insert a rhinoscope or endoscope through the other nostril in order to visualize the treatment site, based on the user's clinical judgment. Alternately, an endoscope or camera may be associated with the shaft 3 to allow direct visualization with the cryogenic gas treatment tool 50. Alternately, the user may actuate the knob 2 to release fluid from the reservoir 7 prior to bending the shaft 3. As another alternative, the user may actuate the knob 2 to release fluid from the reservoir 7 after advancing the distal end of the shaft 3 to the treatment site.

The distal end of the shaft 3 has been advanced to the treatment site. Referring also to FIG. 4, the user visualizes the treatment site. As needed, the user rotates the distal knob 21 to cause rotation of the rotating tip 25 in order to place the distal end 74 of the conduit 18 at the appropriate orientation. As seen in FIG. 10, the rotating tip 25 is oriented to release fluid anteriorly.

The user then actuates the trigger 5. The valve 11 moves downward against the bias of the spring 9, moving the lower O-ring 10a out of the way of the distal end of the proximal cryogenic fluid path 16. Cryogenic fluid is then free to flow out of the proximal cryogenic fluid path 16, through the valve seat 62 between the O-rings 10, and through the conduit 18. The cryogenic fluid preferably remains in a liquid phase through some or all of its transit to the distal end of the conduit 18. If so, the expansion of that liquid upon reaching the end of the conduit 18 and exiting the rotating tip 25 causes that liquid to undergo a phase change to a gas. The expansion of the liquid causes the temperature of the gas to decrease. Advantageously, nitrous oxide is used as the cryogenic fluid. When the nitrous oxide exits the distal end of the conduit 18, it undergoes a phase change to a gas, and its temperature decreases to substantially −89° C. This temperature is low enough to be therapeutically effective, and not so low as to generate negative side effects that may occur from the use of liquid nitrogen. However, nitrogen may be used as cryogenic fluid as desired, as may any other suitable cryogenic fluid. The cold nitrous oxide gas 78 exits the distal end 74 of the conduit 18 and is directed to the treatment site. As one example, the cold nitrous oxide gas 78 may be directed onto tissue in four 5-second increments, with intervals between that allow for at least partial reheating of the treatment site due to blood flow within the patient. After each application of gas, the trigger 5 is released, causing the lower O-ring 10a to block the exit of cryogenic fluid from the reservoir 7.

Referring to FIG. 11, the proximal knob 17 then may be rotated to cause rotation of the rotating tip 25 in order to point the distal end 74 of the conduit 18 to the appropriate orientation. As seen in FIG. 11, the rotating tip 25 is oriented to release fluid anteriorly. When the distal end 74 of the conduit 18 is oriented appropriately, the trigger 5 is actuated, and the treatment site is treated with cryogenic fluid as described above. Next, referring to FIG. 12, the shaft 3 is withdrawn and the shaft 3 is reconfigured with a larger curvature by hand outside the nasal cavity 76. The distal end of the shaft 3 is re-inserted into the nasal cavity 76, and the proximal knob 17 then may be rotated to cause rotation of the rotating tip 25 in order to place the distal end 74 of the conduit 18 at another appropriate orientation. As seen in FIG. 12, the rotating tip 25 is oriented to release fluid superiorly. When the distal end 74 of the conduit 18 is oriented appropriately, the trigger 5 is actuated, and the treatment site is treated with cryogenic fluid as described above. The rotating tip 25 may be placed at other locations requiring treatment in the patient's nasal cavity, and treatment is repeated as described above. When treatment is complete, the shaft 3 is removed from the patient's nasal cavity 76.

As needed from a treatment perspective, shafts 3 may be exchanged intraoperatively one or more times. For example, after treating the patient with the rotating tip 25 as seen in FIG. 10, the shaft 3 may be withdrawn from the patient's nasal cavity 76, removed from the handle 1, and replaced with a different shaft 3, such as one associated with the paddle 23. The shaft 3 is then re-inserted into the patient's nasal cavity 23, and its distal end advanced to a treatment site, where treatment is performed.

For the purposes of describing and defining the present invention it is noted that the use of relative terms such as “substantially,” “generally,” “approximately,” and the like, are utilized herein to represent an inherent degree of uncertainty that may be attributed to any quantitative comparison, value, measurement, or other representation. These terms are also utilized herein to represent the degree by which a quantitative representation may vary from a stated reference without resulting in a change in the basic function of the subject matter at issue.

Exemplary embodiments of the present invention are described above. No element, act or instruction used in this description should be construed as important, necessary, critical or essential to the invention unless explicitly described as such. Although only a few of the exemplary embodiments have been described in detail herein and those skilled in the art will readily appreciate that many modifications are possible in these exemplary embodiments without materially departing from the novel teachings and advantages of this invention. Accordingly all such modifications are intended to be included within the scope of this invention. The phrase “in one embodiment” is used repeatedly. The phrase generally does not refer to the same embodiment; however, it may. The terms “comprising,” “having” and “including” are synonymous, unless the context dictates otherwise. The following illustrations of various embodiments use particular terms by way of example to describe the various embodiments, but this should be construed to encompass and provide for terms such as “method” and “routine” and the like.

Various aspects of the illustrative embodiments will be described using terms commonly employed by those skilled in the art to convey the substance of their work to others skilled in the art. However, it will be apparent to those skilled in the art that the embodiments described herein may be practiced with only some of the described aspects. For purposes of explanation, specific numbers, materials and configurations are set forth in order to provide a thorough understanding of the illustrative embodiments. However, it will be apparent to one skilled in the art that the embodiments described herein may be practiced without the specific details. In other instances, well-known features are omitted or simplified in order not to obscure the illustrative embodiments.

The characteristics and utilities of the present invention described in this summary and the detailed description below are not all inclusive. Many additional features and advantages will be apparent to one of ordinary skill in the art given the following description. There has thus been outlined, rather broadly, the more important features of the invention in order that the detailed description thereof that follows may be better understood, and in order that the present contribution to the art may be better appreciated.

In this respect, by explaining at least one embodiment of the invention in detail, it is to be understood that the invention is not limited in its application to the details of construction and to the arrangements of the components set forth in the description. The invention is capable of other embodiments and of being practiced and carried out in various ways. Also, it is to be understood that the phraseology and terminology employed herein are for the purpose of description and should not be regarded as limiting.

As such, those skilled in the art will appreciate that the conception, upon which this disclosure is based, may readily be utilized as a basis for the designing of other structures, methods and systems for carrying out the several purposes of the present invention. It is important, therefore, that the description be regarded as including such equivalent constructions insofar as they do not depart from the spirit and scope of the present invention.

Further, the purpose of the foregoing abstract is to enable the U.S. Patent and Trademark Office and the public generally, and especially the scientists, engineers and practitioners in the art who are not familiar with patent or legal terms or phraseology, to determine quickly from a cursory inspection the nature and essence of the technical disclosure of the application. The abstract is neither intended to define the invention of the application, nor is it intended to be limiting as to the scope of the invention in any way. The characteristics and utilities of the present invention described in this summary and the detailed description below are not all inclusive. Many additional features and advantages will be apparent to one of ordinary skill in the art given the detailed description.

What is claimed is:

1. A medical apparatus, comprising:
a handle including a proximal knob at the distal end thereof;
a reservoir of cryogenic fluid in said handle;
a shaft extending distally from said handle, said shaft configured to convey cryogenic fluid from said reservoir to a distal end of said shaft; wherein said shaft comprises a malleable material; and
a rotating tip at a distal end of said shaft, wherein rotation of said proximal knob causes rotation of said rotating tip about an axis of said shaft.

2. The medical apparatus of claim 1, wherein said malleable material is annealed steel.

3. The medical apparatus of claim 1, wherein said shaft includes a conduit within a malleable component.

3. The medical apparatus of claim 1, wherein said shaft is detachably connected to said handle.

4. The medical apparatus of claim 1, wherein said shaft further comprises a nozzle at a distal end thereof.

5. The medical apparatus of claim 1, wherein said nozzle is oriented off-axis.

6. The medical apparatus of claim 1, wherein said shaft further comprises a paddle at a distal end thereof.

7. The medical apparatus of claim 1, wherein said shaft further comprises a curved end effector at a distal end thereof.

8. The medical apparatus of claim 1, further comprising a conduit extending from said handle through said shaft to said distal end of said shaft, wherein said conduit conveys cryogenic fluid from said reservoir to a distal end of said shaft.

9. The medical apparatus of claim 1, wherein said shaft further comprises a distal knob at a proximal end thereof; and wherein said distal knob engages said proximal knob to connect said shaft to said handle.

10. The medical apparatus of claim 9, wherein said shaft includes a conduit within a malleable component; and wherein a proximal end of said malleable component is fixed to said distal knob and a distal end of said malleable component is fixed to said rotating tip.

11. The medical apparatus of claim 1, wherein said cryogenic fluid is nitrous oxide.

12. A method for treating tissue of a patient, comprising:
possessing a handle including a proximal knob at the distal end thereof, a reservoir of cryogenic fluid in said handle, a shaft extending distally from said handle, said shaft configured to convey cryogenic fluid from said reservoir to a distal end of said shaft, wherein said shaft comprises a malleable material, and a rotating tip at a distal end of said shaft, wherein rotation of said proximal knob causes rotation of said rotating tip about an axis of said shaft;
apposing said distal end of said shaft to tissue of a patient;
causing cryogenic fluid to travel from said handle to said distal end of said shaft;
orienting said distal end of said shaft to a desired orientation by rotating said proximal knob to cause said rotating tip to rotate about an axis of said shaft; and
expanding said cryogenic fluid to a gas phase upon exit of said cryogenic fluid from said distal end of said shaft, wherein said gas phase cryogenic fluid is at a cryogenically therapeutic temperature.

13. The method of claim 12, further comprising bending said shaft before said apposing.

14. The method of claim 12, wherein said cryogenically therapeutic temperature is substantially −89° C.

15. The method of claim 12, wherein said cryogenic fluid is nitrous oxide.

16. The method of claim 12, wherein said expanding further comprises directing said cryogenic fluid off-axis during said expanding.

17. The method of claim 16, wherein said shaft further comprises a nozzle at a distal end thereof; wherein said directing comprises orienting said nozzle.

18. The method of claim 12, wherein said apposing comprises inserting said distal end of said shaft into a nasal cavity of a patient.

19. The method of claim 12, further comprising, after said expanding, detaching said shaft from said handle and attaching a different said shaft to said handle.

20. A medical apparatus, comprising:
a handle including a proximal knob at the distal end thereof;
a reservoir of cryogenic fluid in said handle;
a shaft extending distally from said handle, said shaft configured to convey cryogenic fluid from said reservoir to a distal end of said shaft; wherein said shaft comprises
a malleable component,
a distal knob affixed to a proximal end of said malleable component, and
a conduit within a malleable component; wherein said distal knob engages said proximal knob to connect said shaft to said handle; and
a rotating tip at a distal end of said shaft, wherein a distal end of said malleable component is fixed to said rotating tip and wherein rotation of said proximal knob causes rotation of said rotating tip about an axis of said shaft.
